(19) United States
(12) Patent Application Publication
(10) Pub. No.: US 2019/0069785 A1
(43) Pub. Date: Mar. 7, 2019

(54) COMBINED TEXTILE PRESSURE AND OPTIC SENSOR

(71) Applicants: FOOTFALLS AND HEARTBEATS (UK) LIMITED, Nottingham (GB); THE UNIVERSITY OF NOTTINGHAM, Nottinghamshire (GB)

(72) Inventors: Simon MCMASTER, Lincolnshire (GB); Barrie HAYES-GILL, Nottinghamshire (GB); Stephen MORGAN, Nottinghamshire (GB); Sergiy KORPOSH, Nottinghamshire (GB)

(73) Assignees: FOOTFALLS AND HEARTBEATS (UK) LIMITED, Nottingham (GB); THE UNIVERSITY OF NOTTINGHAM, Nottinghamshire (GB)

(21) Appl. No.: 15/757,329

(22) PCT Filed: Sep. 5, 2016
(86) PCT No.: PCT/GB2016/052728

§ 371 Date: Mar. 2, 2018

Related U.S. Application Data
(60) Provisional application No. 62/214,274, filed on Sep. 4, 2015.

(51) Int. Cl. A61B 5/024; A61B 5/00; A61B 5/021

(52) U.S. Cl. CPC A61B 5/02438; A61B 5/6802; A61B 2562/0247; A61B 5/021; A61B 5/6826; A61B 5/6843

(57) ABSTRACT

A combined sensor adapted to measure at least one medical or clinical sign is provided. The combined sensor comprises a textile sensor configured so as to determine pressure applied to the combined sensor; and an optical sensor. The optical sensor typically comprises at least one fibre-optic sensor (FOS) and may function as a photoplethysmography (PPG) sensor, optionally a reflectance mode photoplethysmography (PPG) sensor. The combined sensor is able to eliminate motion artefacts caused by movement of a subject wearing the sensor thereby facilitating long-term ambulatory monitoring of subjects.

### TECHNICAL FIELD

The invention relates to sensors that can contact human skin and monitor clinical signs, especially wearable sensors.

### BACKGROUND

An increasingly important area in textile design is that of “intelligent textiles” in which electrical signals representing physiological data are collected from garments and transmitted to remote locations, for example, for monitoring, assessment, and intervention by health care professionals. However, such textile devices are generally not truly “intelligent” textiles, as they comprise solid-state electronics placed in a textile shell and worn as apparel.

Creating textile-based sensor systems that interact with humans or animals is challenging as the sensors need to be capable of measuring clinical signs and physiological parameters accurately and in the correct context. However, such sensors must not be cumbersome or hinder normal-movement and functioning. A significant drawback with most textile based sensor systems is that they fail when the subject undertakes normal movement, such as walking or changing body position. This is due to so-called “motion artefacts” that are introduced into the measurements and can significantly affect measurement thresholds or baselines.

A number of key physiological parameters and clinical signs exist that would be desirable to measure with textile-based sensors that could be worn by the subject under study. The capillary refill time (CRT) is the time taken for blood microcirculation beneath the surface of the skin to refill with blood after having pressure applied and removed. CRT measurement is at present accomplished using either the simple quantitative (finger pressed against skin and time for return of colour counted) or complex quantitative measurement using fibre optic sensors adhered with tape to the appropriate body part. Plantar pressure sensing is either via a fixed pressure pad in a gait analysis lab or more expensive orthotic inserts. There are few devices to allow hourly/daily analysis of relative plantar pressure build up in those with diabetic foot neuropathy. Ambulatory blood pressure monitors are still relatively cumbersome. Blood pressure is a fundamental physiological parameter, a so-called ‘vital sign’ used widely as an indicator of illness and therefore “truly” ambulatory would represent an excellent advance. It is also noted that ambulatory or “at home” monitoring of blood pressure produces results that are at best ambiguous and therefore of limited use to clinicians. Oxygen saturation (SpO2) is monitored periodically via a finger or earlobe device at present.

Hence, it is desirable to provide a sensor that is textile based and that can provide ambulatory monitoring of key physiological parameters and clinical/medical signs.

The present invention has been devised to mitigate or overcome at least some of the above-mentioned problems and disadvantages associated with the prior art.

### SUMMARY OF THE INVENTION

According to an aspect of the present invention there is provided a combined sensor comprising a textile sensor configured so as to determine pressure applied to the combined sensor; and an optical sensor. Typically, the combined sensor is for use in contact with or in the vicinity of a skin surface of a subject.

Suitably the combined sensor is adapted to measure at least one medical or clinical sign, such as at least one vital sign. Typically the medical or clinical sign comprises at least one sign selected from the group consisting of: body temperature; blood pressure; oxygen saturation; capillary refill time (CRT); pulse/heart rate including; and alertness.

According to one embodiment of the invention the textile sensor comprises a knitted sensor.

In a specific embodiment of the invention, the knitted sensor is comprised of an electrically conductive yarn that is knitted into a textile that comprises a plurality of stitches thereby forming a defined stitch pattern, which stitch pattern provides a measurable contact resistance, wherein the measurable contact resistance varies when pressure is applied to the textile sensor. Suitably, the pressure is in the form of applied compression of the textile sensor.

In specific embodiments of the invention the stitch pattern comprises stitches selected from the group consisting of: jersey stitches; tuck stitches; miss stitches; and/or laid-in yarns; as well as any combination thereof. Optionally, the stitch pattern comprises at least 50% of jersey stitches. In this embodiment, the remaining stitches may be comprised of a combination of miss stitches and tuck stitches. Alternatively, the remaining stitches may be comprised of a combination of around 5% miss stitches and around 45% tuck stitches. In a further alternative embodiment the remaining stitches are comprised of a combination of around 10% miss stitches and around 40% tuck stitches. Optionally, in yet further embodiments of the invention the remaining stitches are comprised of either a majority (e.g. greater than half) of miss stitches, or of tuck stitches.

In a specific embodiment of the invention the optical sensor comprises at least one light source. Suitably the light source comprises a light emitting diode (LED). In one embodiment of the invention the optical sensor is a photoplethysmography (PPG) sensor, such as a reflectance mode PPG sensor.

According to a specific embodiment of the invention the optical sensor comprises at least one fibre-optic sensor (FOS). Typically, the FOS comprises at least one optic fibre, suitably the FOS of the invention may comprise a plurality of optic fibres, optionally the FOS may comprise more than three optic fibres.

In a specific embodiment of the invention, the FOS comprises at least a first transmitting fibre having a distal and proximal terminus, wherein the first transmitting fibre is connected to a first light source at its proximal terminus and transmits light from its distal terminus, and

a first receiving fibre having a distal and proximal terminus, wherein the first receiving fibre is connected to a first photodetector at its proximal terminus and receives light at its distal terminus; wherein the distal terminus first transmitting fibre is sufficiently aligned with the distal terminus of the first receiving fibre such that light transmitted from the first transmitting fibre may be received by the first receiving fibre.

In one embodiment of the invention, the distal termini of the first transmitting fibre and the first receiving fibre are separated by an air gap. Suitably, the air gap is at most around 10 mm, typically less than about 10 mm, optionally not more than around 7 mm in length.

According to a further embodiment of the invention, the first transmitting and first receiving fibres are comprised within a single integrated optical fibre, however, the distal termini of the first transmitting fibre and the first receiving fibre are separated by a region of optical fibre in which the external cladding has been removed. Suitably, the region of cladding removal is at most around 10 mm, typically less than about 10 mm, suitably not more than around 7 mm in length.

A second aspect of the invention provides a combined sensor, suitable for use in contact with, or in the vicinity of, a skin surface of a subject, the combined sensor comprising:
a textile sensor,
the textile sensor comprising a knitted sensor, wherein the knitted sensor is comprised of an electrically conductive yarn that is knitted so as to form a textile that comprises a plurality of stitches that define a stitch pattern, which stitch pattern comprises a measurable electrical contact resistance, wherein the measurable electrical contact resistance varies when external pressure is applied to the textile sensor; and
an optical sensor,
the optical sensor comprising a fibre-optic reflectance mode photoplethysmography (PPG) sensor.

A specific embodiment of the invention provides a combined sensor wherein the PPG sensor comprises at least a first transmitting fibre having a distal and proximal terminus, wherein the first transmitting fibre is connected to a first light source at its proximal terminus and transmits light from its distal terminus, and a first receiving fibre having a distal and proximal terminus, wherein the first receiving fibre is connected to a first photodetector at its proximal terminus and receives light at its distal terminus, wherein the distal terminus first transmitting fibre is sufficiently aligned axially or coaxially with the distal terminus of the first receiving fibre such that light transmitted from the first transmitting fibre may be received by the first receiving fibre. Optionally, the distal termini of the first transmitting fibre and the first receiving fibre are separated by an air gap. Suitably, the air gap is at most around 10 mm, typically less than about 10 mm, suitably not more than around 7 mm in length. In an alternative embodiment of the invention, the distal termini of the first transmitting fibre and the first receiving fibre are separated by a region of optical fibre in which the external cladding has been removed. Suitably, the unclad region is at most around 10 mm, typically less than about 10 mm, suitably not more than around 7 mm in length.

A third aspect of the invention provides for sensor as described previously for use in a method of monitoring sporting or task orientated performance in a human or animal subject.

A fourth aspect of the invention provides for a sensor as described previously for use in a method of monitoring clinical signs and/or symptoms in a human or animal patient. A specific embodiment of the invention provides for a sensor as described previously wherein the human patient or animal is suffering from one or more clinical condition or disease selected from the group consisting of: type I or type II diabetes; peripheral vascular disease; cardiovascular disease; kidney disease; hypertension; and cardiac arrhythmia.

A fifth aspect of the invention provides a garment comprising the combined sensor described previously. Optionally, the garment comprises a sock or stocking.

A sixth aspect of the invention provides a wound dressing comprising the combined sensor described previously. Suitably the wound dressing comprises a bandage.

A seventh aspect of the invention provides a method for removing motion artefacts from measurements obtained from a skin surface mounted optical sensor, comprising continually recording applied compression at the site of the a skin surface mounted optical sensor and applying a correction to the measurements so as to normalise the measurements and eliminate motion artefacts. In a specific embodiment of the invention, continual recording of applied compression at the site of the skin surface mounted optical sensor is achieved by combining the optical sensor with a sensor that measures applied compression. Suitably the sensor that measures applied compression is a textile sensor of the type described herein. Optionally, the skin surface mounted optical sensor comprises a FOS as described herein.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

One or more embodiments of the invention will now be described, by way of example only, with reference to the accompanying drawings, in which:

FIG. 1 is a schematic diagram of a combination sensor in contact with a skin surface according to an embodiment of the present invention;

FIG. 2 is a schematic diagram of a cross configuration fibre optic sensor for use in the combination sensor of FIG. 1;

FIG. 3 is a chart illustrating a capillary refill time measurement made using the cross configuration fibre optic sensor of FIG. 2;

FIG. 4a is a diagrammatic view of two interconnected yarn units in a single jersey knit stitch pattern;

FIG. 4b is a diagrammatic view of a plain single jersey knit stitch pattern for use in a textile sensor for use in the combination sensor of FIG. 1;

FIG. 5 is a diagrammatic view of an alternative embodiment of the textile sensor which has a knit stitch pattern having single jersey stitches, miss stitches, and tuck stitches;

FIGS. 6a and 6b are charts illustrating the suitability of a textile sensor of the kind shown in FIG. 5 for measuring pressure;

FIG. 7a is a chart illustrating weight applied to a finger during the capillary oxygen saturation measurement of FIG. 7b measured using a textile sensor of a combination sensor;

FIG. 7b is a chart illustrating a capillary oxygen saturation measurement made using the cross configuration fibre optic sensor of FIG. 2;

FIG. 8 is a chart illustrating a combination measurement made using the combination sensor of FIG. 1 to establish a CRT for a patient;

FIG. 9 is a schematic diagram of a coaxial configuration fibre optic sensor that may be used in a combination sensor;

FIG. 10 is a schematic diagram of a continuous configuration fibre optic sensor that may be used in a combination sensor;

FIG. 11 is a chart illustrating a capillary refill time measurement made using the continuous configuration fibre optic sensor of FIG. 11;

FIG. 12 is a schematic plan view of a combination sensor;

FIG. 13 is a picture of a sole of a foot illustrating positions where a combination sensor may be used to measure physiological parameters of the foot; and

FIG. 14 is a set of combination measurements made on the sole of the foot at the positions shown in FIG. 13.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs.

Prior to setting forth the invention, a number of definitions are provided that will assist in the understanding of the invention.

As used in this description, the singular forms “a,” “an,” and “the” include plural referents unless the context clearly dictates otherwise. Thus, for example, the term “a sensor” is intended to mean a single sensor or more than one sensor or to an array of sensors. For the purposes of this specification, terms such as “forward,” “rearward,” “front,” “back,” “right,” “left,” “upwardly,” “downwardly,” and the like are words of convenience and are not to be construed as limiting terms. Additionally, any reference referred to as being “incorporated herein” is to be understood as being incorporated in its entirety.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

As used herein, the terms “distal” and “proximal” are used to refer to orientation along the longitudinal axis of the apparatus. Since the fibres of the invention are elongate in nature and conform to a single dimension, in use the distal direction refers to the terminus of the fibre furthest away from the source or receiver and the proximal direction to the terminus of the fibre closest to the source or receiver. It should be noted that the term proximal should not be confused with the term ‘proximate’, which adopts its conventional meaning of ‘near to’.

For purposes herein, a “motion artefact” is any error in the perception or representation of a signal introduced by motion of sensor device or a subject to which the device is applied. Motion may be caused by voluntary or involuntary movements of the subject wearing the device of the invention.

As used herein, the term “contact resistance” is used to refer to the total electrical resistance of a portion of the textile due to contacting yarns. The contact resistance varies with the yarn contact area and can change based upon the applied weight or tension applied to the textile. The equation

$${R}_{c} = \frac{ρ}{2} \sqrt{\frac{π H}{F}}$$

is a representation of the Holm contact resistance equation, where R_{c} is contact resistance, ρ is material resistivity, H is material hardness, and F is the normal force. The equation

$${R}_{c} = \frac{ρ}{2} \sqrt{\frac{π H}{nP}}$$

is another representation of the Holm equation, which is more relevant to textile based contact resistance. F is replaced by nP, where n is the number of contact points between adjacent yarn in the textile, and P is the contact pressure. Material hardness and electrical resistivity are constants that depend on the material properties of a textile. Contact resistance is therefore inversely proportional to the number of contact points and the contact pressure. That is, more contact points result in lower contact resistance. Therefore, as the number of contact points and/or contact pressure increases, contact resistance decreases. As used herein, contact resistance provides a measure of electrical conductivity in a yarn or textile. At the “micro” scale, surface roughness limits surface-to-surface contact. In addition, as pressure increases, the number of contact points increases, and eventually at the “nano” scale individual contact points “combine” into a larger contact area. “Integration as Summation” and the “Finite Element Method (FEM)” are techniques that can be used to determine the limits of these contacts points and therefore the contact area they produce.

As used herein, the term “textile” and “fabric” refers to a flexible material manufactured from a plurality of individual fibres that have been combined. A textile or fabric may be woven, knitted, crocheted, spread or made by any other kind of interlacing that may be achieved using fibres. A “fibre” used in relation to a textile refers to any substantially elongate yarn or thread.

As used herein, a “miss stitch” is defined as a knitting stitch in which at least one needle holds the old loop and does not receive any new yarn across one or more wales. A miss stitch connects two loops of the same course that are not in adjacent wales.

For purposes herein, “plain stitch” refers to a knitting stitch in which a yarn loop is pulled to the technical back of a fabric. A plain stitch produces a series of wales or lengthwise ribs on the face of the fabric and courses, or cross-wise loops, on the back. A plain stitch can also be referred to as a “single-knit jersey stitch” or a “single jersey stitch.”

A “tuck stitch” is defined for use herein as a knitting stitch in which a yarn is held in the hook of a needle and does not form a new loop.

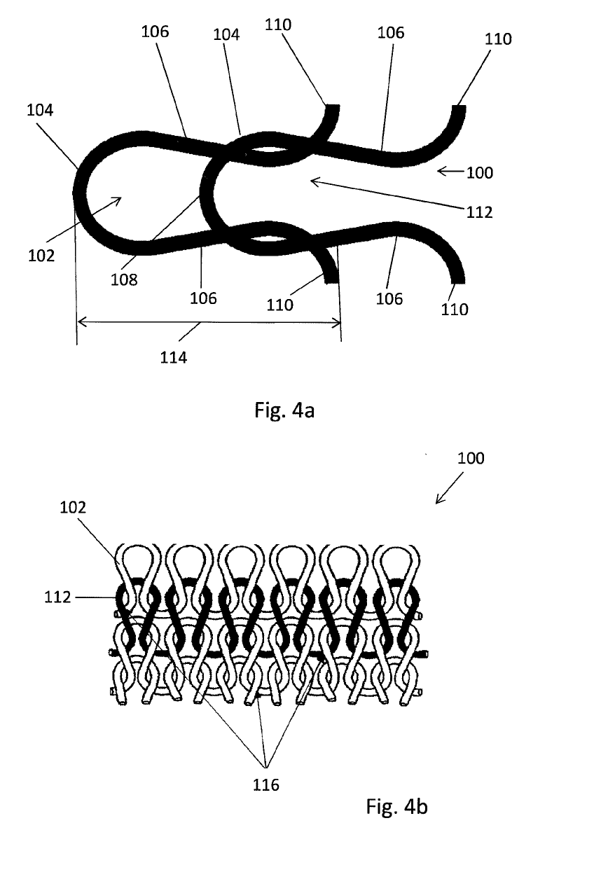

FIG. 4a is a schematic representation of a textile comprising a single jersey stitch 100 and illustrates the concept of yarn contact area. In a jersey knit textile, a needle loop 104, or yarn unit, comprises a head 104 and two side legs 106 that form a noose 108. At the base of each leg 106 is a foot 110, which meshes through the head 104 of a sinker loop 112 formed at the previous knitting cycle. The leg 106 of the needle loop 104 passes from one side (or face) to the other side/face of the sinker loop 112 across the leg 106 and head 104 of the sinker loop 112, and then loops around to pass back across the head 104 and opposite leg 106 of the sinker loop 112 to back to the original side/face of the sinker loop 112. Stitch length 114 is defined as a length of yarn which includes the needle loop 104 and a half of the sinker loop 112 on either side of it.

Yarn contact area is influenced by many different variables of the textile, and has a direct influence on contact resistance of a textile formed of electrically conductive yarns. Contact resistance is associated with the conduction characteristic of the yarn contact surface area. A larger yarn contact area and less surface roughness of the yarn surface results in a lower resistance to electrical signals travelling through the textile. Thus, an increase in yarn contact area causes a proportional decrease in contact resistance. Yarn variables, stitch variables, and textile variables each influence yarn contact area, and thereby provide variables that can be used to specifically design a textile having a yarn contact area, and thus contact resistance, adapted for a particular sensing activity or use.

Variables that can affect contact resistance include: yarn type or composition; yarn fabrication method; yarn count; stitch type, composition, or pattern; stitch length; stitch percentage; mean electrical resistivity (MER); fabric thickness; fabric weight; optical porosity (OP); and percentage permanent stretch (PPS).

FIG. 4b is a schematic drawing of a single jersey stitch pattern. In this pattern, interconnecting stitch loops touch at single jersey contact points 116. In a single jersey stitch pattern, one stitch contacts an adjacent stitch essentially on only one side, or surface, of the adjacent stitch (or fabric) at a time. That is, in two interconnected stitch loops, the legs of a first stitch loop contact the feet of a second, adjacent stitch loop on one surface of the second stitch loop. On the opposite surface of the second stitch loop, the head of the first stitch loop contacts the legs of the second stitch loop. As a result, single jersey contact points are limited to relatively small crossover points of adjacent loops.

As used herein, the term “optical fibre” or “fibre optic” is a flexible, transparent filament through which electromagnetic signals can be communicated. A transparent core of the optical fibre is surrounded by a cladding material around its exterior circumferential surface, the cladding material having a different refractive index to that of the core which ensures that electromagnetic waves reaching the boundary between cladding and core undergo total internal reflection.

The phrase “skin surface” as used herein is intended to refer to the epidermal surface of a subject, typically a human or animal, that is being monitored. In mammals, the skin comprises the outer epidermal layer and the underlying dermis, as well as and supporting tissues including the vasculature associated with the skin.

The present invention provides a combination sensor that comprises a textile incorporating a textile sensor and an optical sensor. The combination sensor is configured for use in direct physical contact with or in the close vicinity of a skin surface of a subject. The combination sensor is configured to measure a sensing activity. In one embodiment of the invention the combination sensor is configured to monitor a single sensing activity. In another embodiment of the invention the combination sensor is capable of measuring and monitoring a plurality of sensing activities concurrently and/or consecutively.

The device of the present invention is suitable for measurement, as well as continuous monitoring, of important physiological parameters and medical/clinical signs (e.g. sensing activities) such as those selected from the non-limiting group comprising: body temperature; blood pressure; oxygen saturation; capillary refill time (CRT); heart rate including variations in normal heart rate (e.g. cardiac arrhythmia); and alertness. It will be appreciated by the skilled reader that the devices and methods of the present invention are not exclusively for diagnostic or prognostic purposes. Measurement of physiological parameters and vital signs (also referred to as “vitals”) may serve multiple purposes, including ongoing monitoring of task-oriented or sporting performance. By way of example, continuous monitoring of astronauts, military personnel or other workers in extreme environments (e.g. deep sea divers) is routine and not exclusively diagnostic in nature.

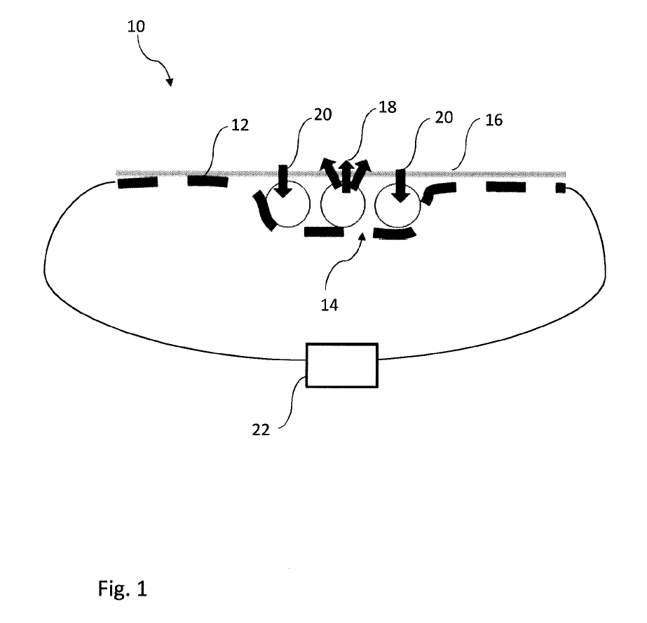

In a specific embodiment, the present invention provides a combination sensor comprising a textile that incorporates at least one textile sensor (TS) and at least one fibre optic sensor (FOS) as seen in FIG. 1. FIG. 1 shows a cross section of a combination sensor 10, the TS 12 is in contact with and applies compression to the FOS 14. In this way the FOS 14 is securely placed against a skin surface 16 of a subject, in this case the sole of the foot of a human patient. Light emitted by the FOS 14 is absorbed 18 or reflected 20 back by the skin surface 16 and blood circulation beneath the skin surface 16. The TS 12 connects to a central control unit 22 which measures the amount of force applied by the textile to the TS 12 and consequently the FOS 14.

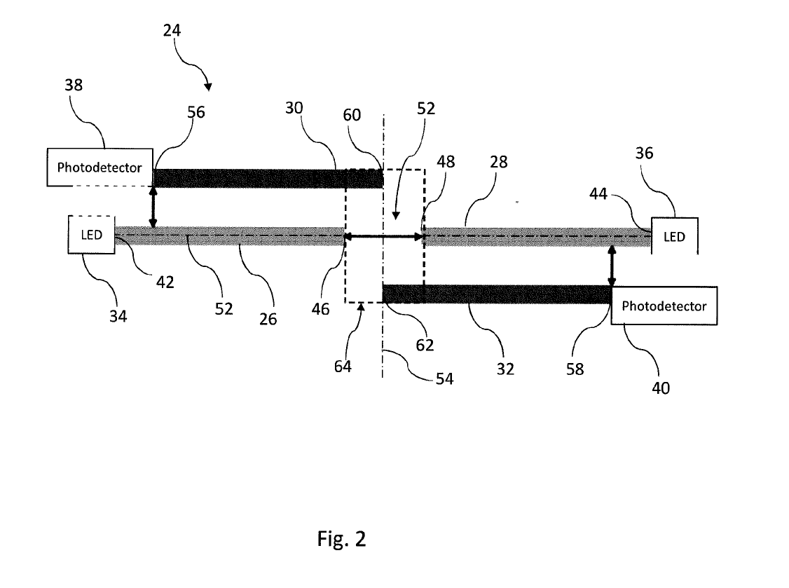

A FOS 14 as shown in FIG. 1 is a reflectance mode photoplethysmography (PPG) sensor, the configuration of which is shown in FIG. 2. FIG. 2 shows a plan view arrangement of part 24 of the FOS 14 in FIG. 1, and the textile sensor 12 is not included in FIG. 2 for clarity. The FOS 24 of FIG. 2, which shall hereinafter be referred to as the cross configuration FOS 24 to distinguish from other possible optical sensor configurations, incorporates a first and second transmitting optical fibre, hereafter referred to as the left transmitting optical fibre 26 and a right transmitting optical fibre 28; and a corresponding first and second receiving optical fibre, hereafter referred to as the left receiving optical fibre 30, and the right receiving optical fibre 32. As is convention for optical fibres, an outer surface of the fibre is coated with cladding to ensure that total internal reflection occurs along the length of the optical fibre, thereby reducing any potential loss of signal or introduction of noise into the signal.

The cross configuration FOS 24 also includes a light source or sources such as first and second (left and right) light emitting diodes 34 and 36 (LEDs) and a corresponding first and second receiver or receivers such as left and right photodetectors 38 and 40. Each LED 34, 36 connects to a proximal terminus 42, 44 of its respective transmitting fibre 26, 28, and transmits light to a distal terminus 46, 48 of that transmitting fibre 26, 28. Each terminus 42, 44, 46, 48 is formed by cutting or cleaving the fibre 26, 28 to form a transverse surface that may be angled at around 45 degrees to the longitudinal axis. The surface of the terminus 42, 44, 46, 48 may then be polished to facilitate optimal light transmission. The distal termini 46, 48 of the transmitting fibres 26, 28 are arranged coaxially to lie opposite each other, spaced apart at a distance such that an air gap 50 is formed between the two distal termini 46, 48 of the transmitting fibres 26, 28. The fibres 26, 28 are therefore aligned along a longitudinal first axis 52 when the cross configuration FOS 24 is laid flat, and arranged at a specific distance from and on either side of a central axis 54 that is perpendicular to the longitudinal axis 52 along which the transmitting fibres 26, 28 lie.

Similarly, each photodetector 38, 40 connects to a proximal terminus 56, 58 of its respective receiving fibre 30, 32, and receives light from a distal terminus 60, 62 of that receiving fibre 30, 32. The distal termini 60, 62 of the receiving fibres 30, 32 lie on the central axis 54, the fibres 30, 32 extending away from the central axis 54 in opposite directions. The fibres 30, 32 are aligned in parallel with the transmitting fibres 26, 28, at least in the vicinity of the distal termini 60, 62 of each fibre 30, 32, and are offset from the transmitting fibres 26, 28.

By offsetting the receiving fibres 30, 32 from the transmitting fibres 26, 28 the air gap 50 between the transmitting fibres 26, 28 defines a sensing area 64 between the fibres 26, 28, 30, 32. In use, a light signal is communicated along each of the transmitting fibres 26, 28 by its respective LED 34, 36 towards the sensing area 64. As the proximal and distal termini of each fibre are formed by cutting and polishing the fibre, the termini are not covered in cladding, thereby allowing light ingress and egress. For each transmitting fibre 26, 28, the light signal enters the proximal terminus 42, 44 of the fibre 26, 28 and travels along the transmitting fibre 26, 28 by the mechanism of total internal reflection. At the distal end 46, 48, the light exits the fibre 26, 28 and is transmitted into the sensing area 64 which may be adjacent to the skin surface of the subject. Reflection and/or absorption affects the amount of light able to enter the distal termini 60, 62 of the receiving fibres 30, 32. The light that does enter the receiving fibres 30, 32 is totally transmitted through the fibre 30, 32 until it reaches the photodetector 38, 40, where the signal intensity is measured. By only allowing light egress at the sensing area 64, information loss is minimised and a higher signal-to-noise ratio (SNR) achieved.

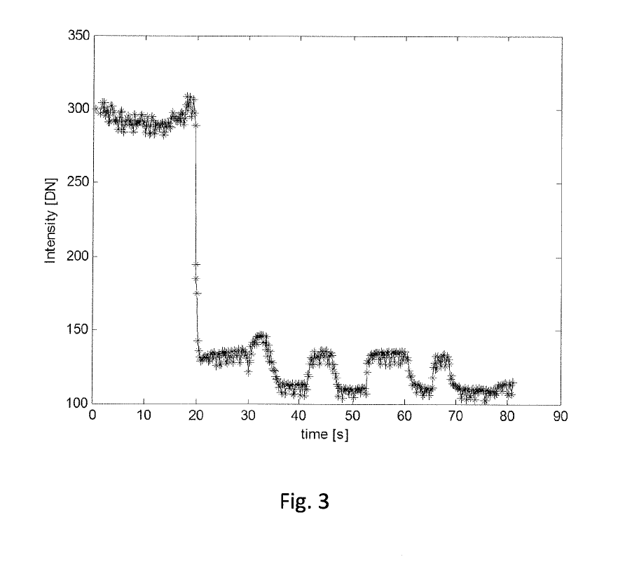

The efficacy and capability of the cross configuration FOS 24 is illustrated by FIG. 3. FIG. 3 was obtained by measuring capillary refill time (CRT) in the skin of a patient to whom the sensor 24 was applied. When measured with the cross configuration FOS 24 as shown in FIG. 3, pressure in the form of compression is applied at regular intervals, and removed. When the pressure is applied to the sensor 24 and the skin surface 16, the intensity of the light measured by the photodetectors 38, 40 increases due to increased reflection of light from the transmitting fibres 26, 28 to the receiving fibres 30, 32. When the pressure is removed, the intensity drops, until settling at a baseline level where the skin is fully reperfused with blood. Following each application and removal of pressure to the sensing area, the intensity of the measured light reduces to a consistent baseline level, with the time taken between the time point at which the pressure is removed and time of return to the baseline level reading corresponds to the CRT. In FIG. 3, the CRT is shown to be approximately 2 s. However, in order to simulate normal ambulatory movement the pressure applied each time differs, resulting in different peaks in the measurement, and therefore, potentially different CRTs. To alleviate such artefacts, the combination sensor incorporates a cross configuration FOS and a TS. In this way, the sensor of the invention allows for the measured light intensity to be normalised with respect to pressure applied (as determined by the TS) and therefore, an accurate CRT can be obtained. Additionally, the inclusion of a pressure sensing TS allows the CRT to be measured only when a predefined threshold pressure is exceeded. The combination sensor configuration of the invention, therefore, allows the sensor to be truly ambulatory which enables continuous monitoring of the subject throughout their normal activities. This is of considerable advantage in that in clinical settings it allows patients to continue their day-to-day affairs with minimal impact or hindrance. In non-clinical studies, such as in assessment of sporting performance, freedom and range of movement is minimally compromised, if at all. Clearly, this leads to greater accuracy of real-life measurements that has, hitherto, not been feasible using prior art sensor arrangements.

It has been found that the modulation depth of the CRT measurement between the peaks and troughs of the intensity level are proportional to the absolute blood volume of the circulation in question.

According to one embodiment of the present invention, the TS is comprised within a specific zone of a textile and is fabricated from electrically conductive yarn. The TS is typically a fully integrated knitted sensor within the textile, which itself may form a garment, the sensor having been designed and adapted for a sensing activity such as for sensing applied pressure and/or compression. The TS may be knitted and comprises a plurality of stitches forming a stitch pattern. The plurality of stitches may comprise any combination of jersey stitches, tuck stitches and miss stitches or laid-in yarns. An example stitch pattern 100 is shown in FIG. 4b, where a stitch pattern 100 comprising 100% jersey stitches is shown. A suitable textile sensor of this kind is the subject of further patent applications for applicant Footfalls and Heartbeats Ltd, with application numbers PCT/162014/058866 and PCT/162014/063929.

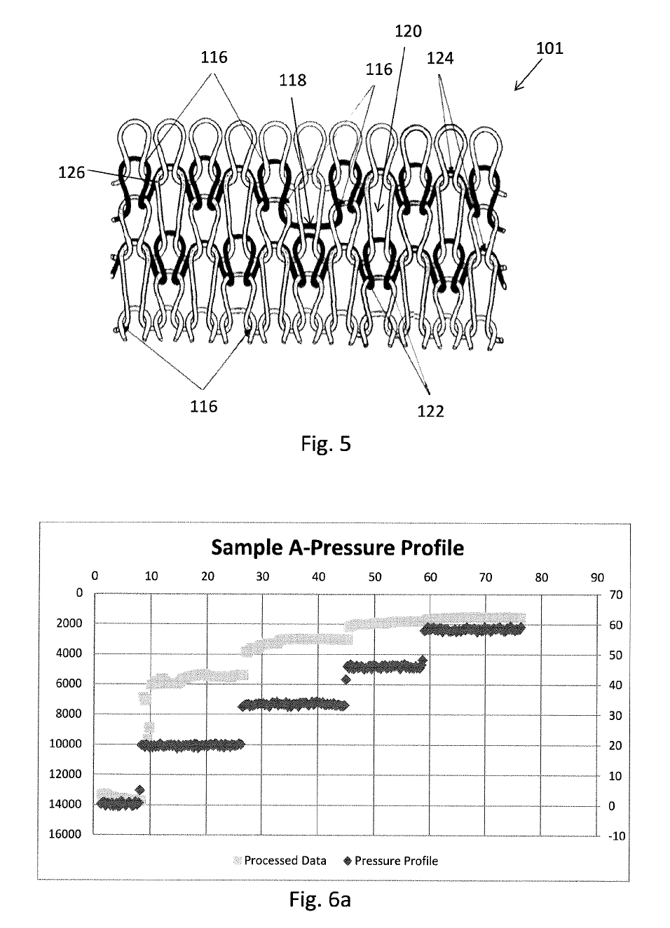

FIG. 5 is a schematic drawing of a single jersey stitch pattern 101 having miss and tuck stitches, which is an alternative embodiment of the TS that may be used in combination with an optical sensor, such as a FOS, as described herein. A single jersey stitch pattern 101 having miss and tuck stitches includes single jersey contact points 116, as well as additional contact points at the miss 118 and tuck stitches 120. A tuck stitch contact point 122 occurs when a tuck stitch loop interconnects in a course with adjoining stitch types. A tuck loop contact point 124 occurs when the tuck loop of a tuck stitch presses upon the held loop of a tuck stitch. A held loop contact point 126 is formed when the held loop of a tuck stitch is forced against an adjacent stitch loop.

As compared to the plain single jersey stitch pattern 100 seen in FIG. 4b, the different contact points and areas shown in the tuck stitch and miss stitch structures in FIG. 5 allow for different contact areas between textiles having different stitch patterns, and therefore a predictable contact resistance that can be designed specifically for a given application or sensing activity.

Alternative wearable sensors having the ability to detect and determine applied external forces, such as compression, may be used in the combination sensor of the invention without departing from the scope of the claims set out below.

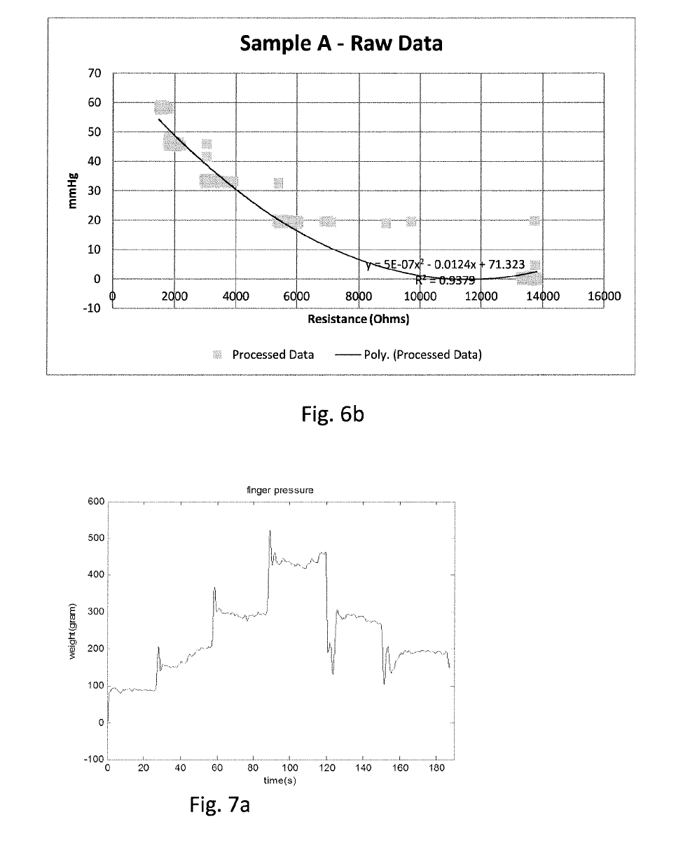

FIGS. 6a and 6b show one embodiment of the invention and, in particular, how the TS is able to measure pressure applied to the sensor both within quantifiable ranges and as raw data. The TS is a knitted textile that incorporates electrically conductive yarn with a configuration of 50% jersey stitches, 5% miss stitches and 45% tuck stitches. FIG. 6a is graph showing the processed data from a TS alongside a commercially available ‘Flexiforce’ pressure sensor by way of comparison. Increasing levels of pressure were applied over time to both the TS and the Flexiforce sensor, with measurements being taken to establish the relationship between contact resistance in ohms (Ω) within the textile (shown on the left hand axis) and pressure (right hand axis) in mmHg.

To briefly elaborate on FIG. 6a, the reading taken between 10 s and 25 s, for example, corresponds to a pressure of 20 mmHg taken by the commercial pressure sensor, and to approximately 5500Ω measured in the textile.

This measurement demonstrates that there is a clear relationship between pressure and contact resistance in the TS, and allows a polynomial relationship between resistance and pressure to be established, which is illustrated in FIG. 6b.

In the combination sensor, the FOS is appropriately fixed in position relative to the TS. The FOS is fixed using a fixing band that connects each FOS to the textile or TS. Alternatively, a sensor plate may be incorporated into the TS, such that the TS entirely surrounds the sensor plate. The FOS is then attached to the sensor plate by a fixing band or adhesive.

In an alternative embodiment, the FOS is laid into a channel formed in the textile. The termini of the fibres are exposed to the skin surface at the TS, and are not laid into the TS. In alternative embodiments, the FOS is laid into the textile and the TS. In such a configuration, the textile structure can help hold the FOS in a desired position and allows control of the dimensional stability of components of the FOS. Such an arrangement minimizes the potential for interference by motion of the FOS and the sensing area relative to each other, and the potential for interference on measurement accuracy of the FOS. In addition, holding a FOS in position in a TS structure and in the textile structure around the textile sensor can help avoid “kinks” in the optical fibres of the FOS which cause problems and lower the lifespan of the sensor.

Now considering a CRT experiment carried out using a combined sensor as described above, the source and receiver of the FOS and the TS may be connected to a central control unit (not shown) such as a processor. The processor may be incorporated into the combination sensor or may be external to it, in a mobile device (such as a smart phone) for example, communicated with via a wireless protocol and exchange module. The processor is configured to implement and record the measurement. During the CRT measurement, the processor will record the time taken for the measured light intensity to return to a predetermined baseline level at which the skin is perfused with blood following an application of pressure. The pressure will be measured and any motion artefacts accounted for by the processor. The processor is configured to determine the capillary refill rate from the output of the light detector.

It is well known that the capillary refill rate may show a substantially linear temperature dependency, and the temperature of the illuminated region (or a region nearby) may thereby be used to provide temperature compensation (for example by means of a lookup table). This may be achieved using the TS or a separate TS incorporated into the textile. Alternatively, a reference fibre or thermocouple incorporated into the textile provides temperature compensation and other reference information. In one embodiment, the reference fibre is completely cladded and not used for measurement. Instead, a signal transmitted along the reference fibre is compared to known values and parameters of the external environment are established from the comparison, such as any variation in temperature. A reference fibre may also be incorporated to account for external lighting conditions and changes that may cause changes in received light.

The output of the pressure sensing TS may be used to trigger the timing of the capillary refill measurement, and/or the capillary refill measurement may be corrected based on the magnitude and/or duration of loading prior to unloading. In this manner, the combination sensor is able to provide an ambulatory sensor that continuously operates. If the combination sensor is used to measure microcirculation of the sole of the foot, ordinary walking of a wearer can trigger measurements to be made. Measurements of the same pressure can be made each time, thereby normalising the measurement and ensuring that a truly repeatable measurement is possible. In addition, as walking or any pressure and removal of pressure on the sole of the foot may cause a measurement to trigger, many CRT measurements can be used to form a mean, precise value.

In specific embodiments of the invention, the processor performs additional measurement steps and undertakes analysis of the measured data. In other embodiments, the processor varies the output of the light source to provide a higher signal to noise ratio. For example, if the ambient lighting conditions are particularly bright, then the intensity of the light source is increased to ensure that the baseline threshold of light is increased.

In embodiments of the invention, the processor is in communication with a power source. The power source is electrically connected to the source and receiver of the FOS and to the TS. In some embodiments, the power source is electrically connected to the source and/or receiver via the TS or via another electrically conductive yarn or yarns incorporated in the textile.

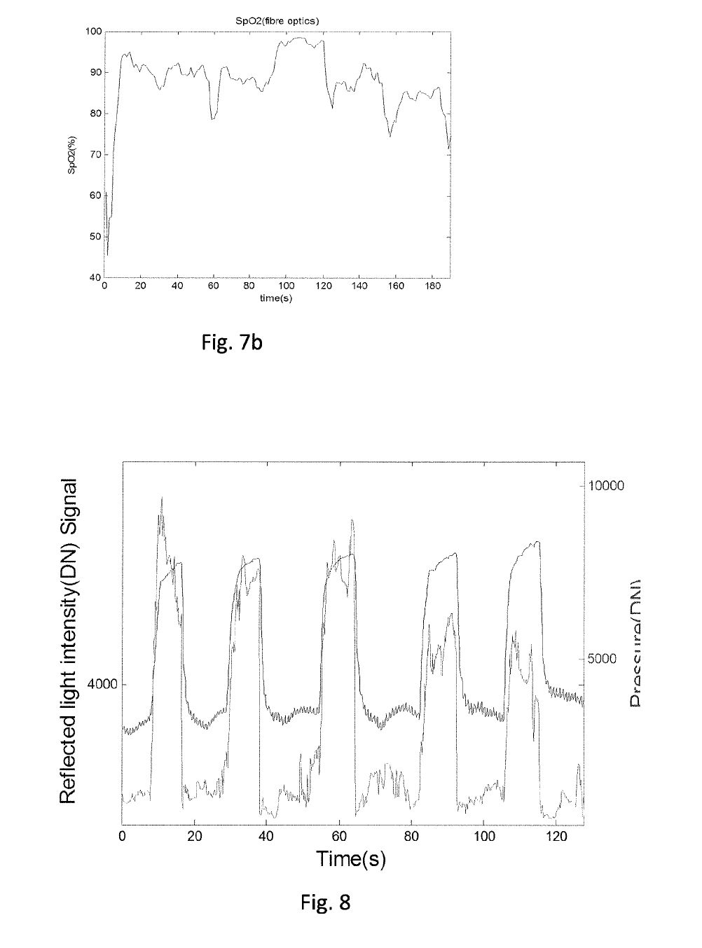

To further illustrate the operation of a combination sensor in use, the cross configuration FOS was used to measure capillary oxygen saturation (SpO₂) of a finger of a patient. The results are illustrated in FIGS. 7a and 7b. The SpO₂ is measured with red and infrared light using photoplethysmography (PPG). The PPG waveform comprises a pulsatile (“AC”) physiological waveform attributed to cardiac synchronous changes in the blood volume with each heartbeat, and is superimposed on a slowly varying (“DC”) baseline with various lower frequency components attributed to respiration, sympathetic nervous system activity, and thermoregulation.

FIG. 7a represents the effect of application of different weights (causing different levels of compression) resulting from increasing pressure applied to the finger at regular 30 second intervals. FIG. 7b shows measurements made by the cross configuration FOS. As shown in FIGS. 7a and 7b, sudden increases in weight and therefore pressure applied to the finger caused by motion, such as those shown at times 30 s, 60 s, 90 s, 120 s and 150 s, correspond to sudden decreases in measured SpO₂ percentage which are designated as motion artefacts. By combining the knowledge of these motion artefacts and the SpO₂ percentages, motion artefacts can be removed from the measurements leading to greatly increased accuracy and true ambulatory monitoring.

In addition, it can be seen that the SpO₂ level rises between 90 s and 120 s to an SpO₂ level that indicates that a threshold pressure has been applied to the finger. Above the threshold, the response of the FOS becomes inaccurate. Therefore, if a maximum pressure threshold is exceeded during use of the combination sensor, the SpO₂ levels measured during the period of exceedance is discounted.

Conversely, a minimum pressure threshold must be exceeded for a measurement to be recorded. It can be seen that between approximately 0 s and 10 s the minimum threshold is not met, resulting in an incorrect measurement. Hence, there is an optimum range of pressures at which SpO₂ can be measured and this is recognised and compensated for by the combinatorial sensors of the type described herein.

In addition, a combination measurement of a CRT measurement made using a combination sensor using a similar methodology as in the above SpO₂ measurement is shown in FIG. 8. Pressure applied to a patient's finger is measured in the right hand Y axis, while the intensity of reflected light is illustrated on the left hand Y axis. Time is measured by the X axis. It can be seen in FIG. 8 that the light intensity changes in response to the applied pressure.

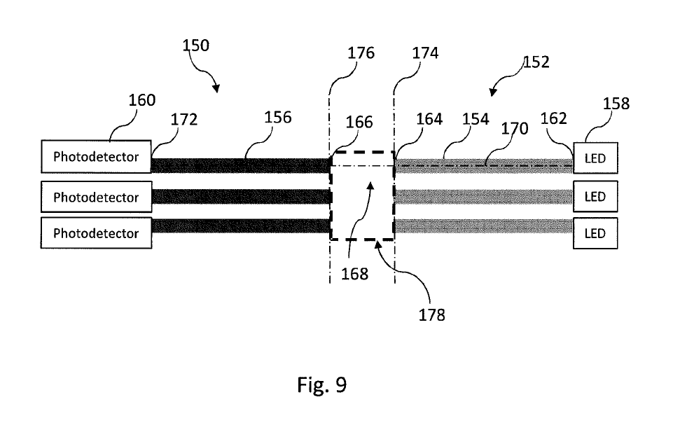

FIG. 9 shows an alternative configuration of a FOS 150 that may be used in the combination sensor 10. The FOS 150 of FIG. 9, known as the coaxial configuration FOS 150 hereinafter, comprises pairs of fibres 152, each pair 152 having a transmitting fibre 154 and a receiving fibre 156. Three fibre pairs 152 are shown in FIG. 9, although more or fewer pairs may be incorporated according to the intended usage. Each pair of fibres 152 is identical, so only a single pair will be described here.

The coaxial configuration FOS 150 also includes a light source for each pair of fibres such as respective light emitting diodes (LED) 158 and a receiver or respective receivers such as a photodetector 160 for each pair of fibres 152. As with the cross configuration FOS 24, each LED 158 connects to a proximal terminus 162 of its respective transmitting fibre 154, and transmits light to a distal terminus 164 of that transmitting fibre 154. Each terminus 162, 164 is formed by cutting the fibre 154 to form a surface angled at 45 degrees to the longitudinal axis of the fibre 154. The surface is then polished. The distal terminus 164 of each transmitting fibre 154 is coaxially arranged to lie opposite the distal terminus 166 of its respective receiving fibre 156, spaced apart at a distance such that an air gap 168 is formed between the two distal termini 164, 166 of the fibres 154, 156. The fibres 154, 156 are therefore aligned along a longitudinal axis 170 when the coaxial configuration FOS 150 is laid flat.

Similarly, each photodetector 160 connects to a proximal terminus 172 of its respective receiving fibre 156, and receives light from a distal terminus 166 of that receiving fibre 156.

Each of the transmitting and receiving fibres 154, 156 comprises cladding along their respective lengths to ensure total internal reflection except for at the cut distal ends 164, 166. The pairs of fibres 152 are arranged in the same orientation and arranged in parallel to each other. Therefore the distal termini 164 of the transmitting fibres 154 are aligned along an axis 174, with the transmitting fibres 154 extending away from the axis in the same direction. Similarly, the distal termini 166 of the receiving fibres 156 are aligned along another axis 176, the receiving fibres 156 extending away from that axis 176 in parallel and in the same direction. Having three pairs of fibres 152 arranged parallel to one another increases the size of a potential sensing area 178. Increasing the number of fibres also means that more scattered light may be detected, which will be discussed later.

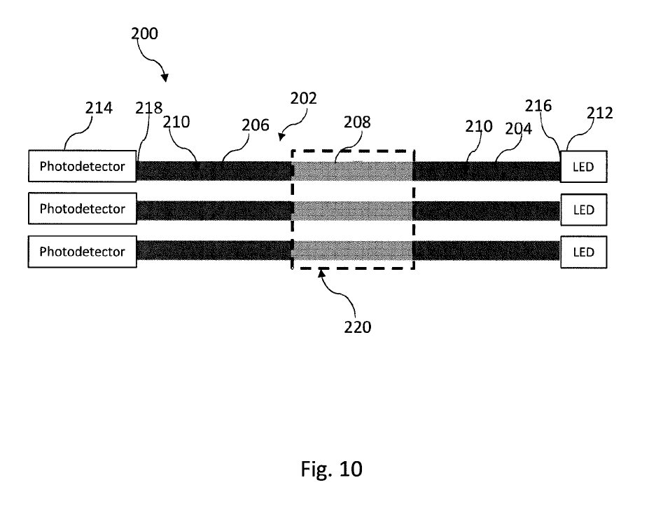

An alternative configuration of a FOS 200 is shown in FIG. 10. The FOS 200 of FIG. 10, known as the continuous configuration FOS 200 hereinafter, comprises one or more optical fibres 202 arranged in parallel, each optical fibre 202 having a transmitting portion 204, a receiving portion 206 and a sensing portion 208. Three fibres are shown in FIG. 10, although more or fewer fibres (e.g. one or two) may be incorporated according to the intended usage. Each of the fibres is identical, so only a single fibre 202 will be referred to here.

As is convention for optical fibres, an outer surface of the fibre is coated with cladding 210 to ensure that total internal reflection occurs along the length of the optical fibre, thereby reducing any potential loss of signal or introduction of noise into the signal. In the previous embodiments of FIG. 2 and FIG. 9, light exchange within the sensing area 64, 178 was enabled creating an air gap 52, 168 between fibres. In the continuous configuration FOS 200, no distal terminus is formed by the fibre 202, and the fibre 202 is continuous from source 212 to receiver 214, the source 212 and receiver 214 being disposed at either terminus 216, 218 of their fibre 202. In this configuration 200, the cladding 210 is removed from the fibre 202 at the sensing portion 208, so as to expose some of an internal core of the fibre to an external environment.

Therefore, in the continuous configuration FOS 200, light travels along the transmitting portion 204 by total internal reflection. At the sensing portion 208 (which corresponds to the sensing area 64, 178 of earlier embodiments), the light is permitted to ‘leak’ out of the fibre 202 into the adjacent skin surface of the subject. Reflection of the light or absorption of the light within the skin and underlying tissue is then measured by the amount of light that returns into the fibre 202 at the sensing portion 208 and travels along the cladded receiving portion 206 to the receiver 214.

The continuous configuration FOS 200 embodiment shown in FIG. 10 comprises three fibres arranged in parallel and spaced approximately apart, again defining a larger sensing zone 220 than would be possible with fewer fibres.

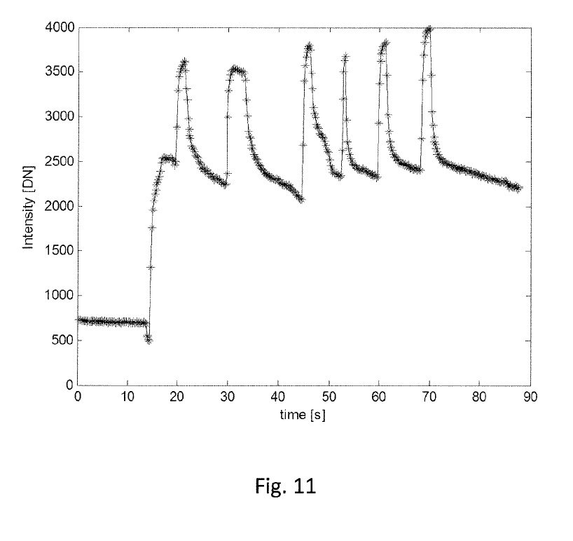

FIG. 11 illustrates a measurement of CRT made using the sensor configuration 200 of FIG. 10.

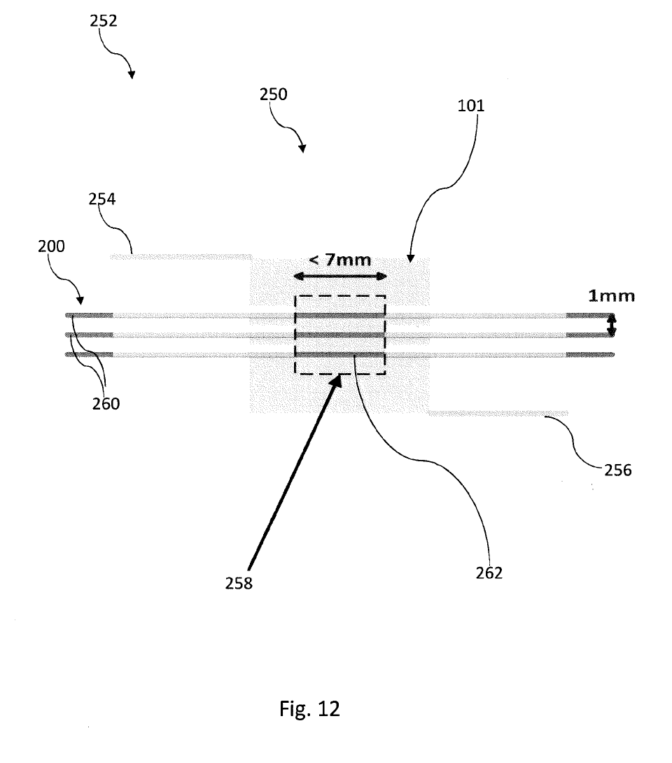

The modulation in the intensity shown in FIG. 12 illustrates that when disposed in air between 0 s and 15 s of the measurement, much less of the signal is reflected back to the receiving fibre than is reflected during the measurement period after this time period. During the measurement period, much more of the light is reflected and it is absorption of the light by blood perfused skin that causes the drop in intensity that corresponds to CRT.

In FIG. 12, the continuous configuration FOS 200 is combined with a textile 250 incorporating a textile sensor 101 of the type shown in FIG. 4b, thereby forming a combination sensor 252. While the continuous configuration FOS 200 is illustrated here, any of the previously detailed configurations of the FOS 24, 150 or any other configuration of the FOS may be used in the same manner with a textile sensor 101 as previously indicated.

The textile 250 has two ‘lead’ regions 254, 256 comprising electrically conductive yarn knitted into the textile 250 which connect the TS 101, and therefore the sensing zone 258, to a central control unit (not shown) and to a power source (not shown).

The optical fibres 260 of the FOS 200 are disposed approximately 1 mm apart, and are laid into the textile 250 so that their position is easily maintained relative to the TS 101. The uncladded sensing portions 262 of the optical fibres 260 are not laid into the textile 250, so that maximum contact can be made with a skin surface, and are arranged to lie at the centre of the TS 101, in a sensing area 258 that is less than about 7 mm across along the axis of the optical fibres 260. According to one embodiment of the invention a sensing zone 258 of less than about 7 mm in the axial direction ensures minimum movement of the optical fibres 260 when the subject is walking.

In alternative embodiments, a sensing zone 258 is greater than about any one of 1 mm, 2 mm, 3 mm, 4 mm, 5 mm, 6 mm, 7 mm, 8 mm, 9 mm, up to 10 mm, and up to 20 mm across may be used. In each case, the TS 101 is configured to be particularly sensitive to the relative position of the optical fibres 260. A large TS 101 may be used for better spatial averaging of the data.

The above described configurations of fibre optic sensors are able to measure a range of physiological parameters including capillary refill time (CRT), capillary oxygen saturation (thereby allowing the combination to be used in pulse oximetry), plantar pressure, heartrate and heartrate variability, and blood pressure.

While the operation of the fibre optic sensors with relation to many of the potential physiological applications is similar to the method described above for measuring CRT and SpO₂, the FOS configurations above can be applied to techniques that detect and process the fluctuating speckle pattern of light reflected from tissue, such as laser Doppler flowmetry (LDF) and laser speckle contrast measurements. These techniques are used to monitor blood flow and pressure.

Microcirculation, and hence the LDF signal, is greatly affected by the pressure exerted on the tissue. Combined pressure and LDF measurements are useful for making clinically relevant measurements for microvascular testing, for example, for post occlusive reactive hyperaemia. Hence, the combined sensor of the present invention allows for monitoring to take place taking account of motion artefacts and the pressure applied. The correct pressure can therefore be regulated and kept constant, and measurements will only be taken above a known pressure threshold, ensuring that no anomalous or imprecise results are achieved.

In LDF, coherent light (usually from a laser) illuminates tissue. Light that is scattered by moving red blood cells undergoes a Doppler frequency shift and interferes with light that is scattered by static tissue (without a Doppler shift) which provides a frequency spectrum between ˜20 Hz-20 kHz. This frequency spectrum is directly detected by a photodiode and then processed to provide an indication of blood flow using an equation of the form:

Flow=M₁=∫_{ω}_{₁}^{ω}^{₂}ωP(ω)dω/DC²,

where M₁ is the first moment of the power spectrum power density spectrum P(ω), ω is the angular frequency of the detected light, and DC is the detected DC light level.

The properties of the detected speckle pattern, and hence the blood flow signal, are also affected by the distance between the sensor and the skin surface. Similarly, monitoring sensor proximity with a TS allows more desirable positioning of the sensor and thus more accurate readings.

The combined sensors of the present invention may be incorporated into garments, wound dressings, bandages, strapping, fabric strips or webbing as appropriate for the desired application. In alternative embodiments of the invention, the combined sensors may be comprised within devices, furniture, surfaces or tools that are designed to come into contact with the skin of a subject but not necessarily worn by said subject. By way of example, combined sensors may be incorporated into vehicle seats or steering apparatus used in motor vehicles or aircraft.

An additional benefit of using a textile pressure sensor is that it can also be used as an indicator of proximity to ascertain when the detector is in contact with the skin surface in order to reduce the effects of motion artefact. This enables the sensor to be worn in loose fitting clothing rather than attached to the skin surface.

Alternatively, blood pressure monitoring can be achieved by measuring a pulse transit time. To achieve this, a PPG measurement is made at 2 different locations on the body such as at an area of an arm and a fingertip of a patient or at a lobe of an ear of a patient and a fingertip. A time of arrival of a pulse at each detector is measured and the arrival time difference can be related to blood pressure.

A number of alternative embodiments are possible without departing from the scope of the invention as claimed. For example, the textile or TS is woven or otherwise fabricated in other embodiments. In some embodiments, a plurality of optical fibres forming a single FOS attach to a single light source, and a single receiver.

In alternative embodiments, a transmittance mode fibre optic sensor is included. A transmittance mode sensor transmits light through a finger or other body part to a receiving fibre disposed on an opposite side. The measurement is made by measuring the transmitted light rather than reflected light.

The invention is further illustrated by the following non-limiting example.

### Example 1—Sock Incorporating the Combination Sensor to Create an Ambulatory CRT Measurement Device for the Sole of a Wearer's Foot

A combination sensor is incorporated into a sock manufactured from a form-fitting textile. The combination sensor monitors physiological parameters of the sole of a wearer's foot. Wearers are particularly at risk people who may suffer from diabetic foot ulcers. This is useful in diagnosing and monitoring the onset of diabetic foot ulcers. Such a sock can also be used in place of conventional pedobarography equipment and to determine efficacy of plantar pressure relieving orthotics.

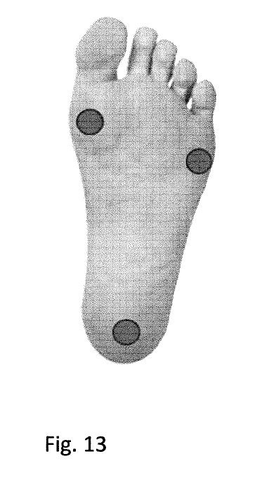

The fabric of the sock comprises the textile sensor, while the fibre optic sensor is laid into the sock accordingly. A coaxial configuration FOS is incorporated to be in contact with the wearer's foot at the points illustrated in FIG. 13. The coaxial configuration FOS each comprises a single pair of plastic optical fibres having a diameter of 500 μm. The first sensing area is in the region of the first metatarsal, the second sensing area is in the region of the fifth metatarsal, and the third sensor area is in the region of the central heel.

A processor and the light source(s), receiver(s) and electrical source(s) are incorporated into the sock so as to be above the wearer's ankle. The wearer walks normally whilst wearing the sock. The textile sensor in each of the three positions can be used to analyse the gait of the wearer, whilst also measuring the pressure applied by the wearer to each sensing position during walking. If the pressure measured exceeds a threshold pressure, a measurement of CRT can be taken between an earlier established baseline and the threshold pressure.

The textile sensor monitors the position of the optical fibres relative to the sensing position and alerts the user if the sock is not in the correct position on the foot. The textile sensor can measure continuously provided that wearer is walking. In times when the wearer is not walking, the pressure is still be monitored to alert the user to any swelling. If swelling is occurring, the user is prompted using a remote device to walk about to enable a measurement of CRT or other functions to identify why the swelling has occurred.

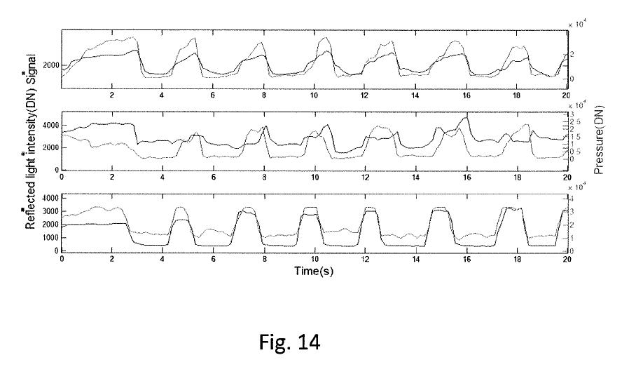

Combination measurements made using the combination sensor are illustrated in FIG. 14. The measurements shown are ordered to relate to the first, second and third sensing areas respectively.

Although particular embodiments of the invention have been disclosed herein in detail, this has been done by way of example and for the purposes of illustration only. The aforementioned embodiments are not intended to be limiting with respect to the scope of the invention. It is contemplated by the inventors that various substitutions, alterations, and modifications may be made to the invention without departing from the spirit and scope of the invention.

1. A combined sensor comprising:
a textile sensor configured so as to determine pressure applied to the combined sensor; and
an optical sensor.

2. The combined sensor of claim 1, wherein the sensor is adapted to measure at least one medical or clinical sign.

3. The combined sensor of claim 2, wherein the medical or clinical sign comprises at least one vital sign.

4. The combined sensor of claim 2, wherein the medical or clinical sign comprises at least one sign selected from the group consisting of: body temperature; blood pressure; oxygen saturation; capillary refill time (CRT); pulse/heart rate including; and alertness.

5. The combined sensor of claim 1, wherein the combined sensor is for use in contact with or in the vicinity of a skin surface of a subject.

6. The combined sensor of claim 1, wherein the textile sensor comprises a knitted sensor.

7. The combined sensor of claim 6, wherein the knitted sensor is comprised of an electrically conductive yarn that is knitted into a textile that comprises a plurality of stitches thereby forming a defined stitch pattern, which stitch pattern provides a measurable contact resistance, wherein the measurable contact resistance varies when pressure is applied to the textile sensor.

8. The combined sensor of claim 7, wherein the pressure is in the form of applied compression of the textile sensor.

9. The combined sensor of claim 7, wherein the stitch pattern comprises stitches selected from the group consisting of: jersey stitches; tuck stitches; miss stitches; and/or laid-in yarns; as well as any combination thereof.

10. The combined sensor of claim 1, wherein the optical sensor comprises at least one light source.

11. The combined sensor of claim 10, wherein the light source comprises a light emitting diode (LED).

12. The combined sensor of claim 1, wherein the optical sensor is a photoplethysmography (PPG) sensor, optionally a reflectance mode photoplethysmography (PPG) sensor.

13. The combined sensor of claim 1, wherein the optical sensor comprises at least one fibre-optic sensor (FOS).

14. The combined sensor of claim 13, wherein the FOS comprises:
at least a first transmitting fibre having a distal and proximal terminus, wherein the first transmitting fibre is connected to a first light source at its proximal terminus and transmits light from its distal terminus; and
a first receiving fibre having a distal and proximal terminus, wherein the first receiving fibre is connected to a first photodetector at its proximal terminus and receives light at its distal terminus;
wherein the distal terminus of the first transmitting fibre is sufficiently aligned axially or coaxially with the distal terminus of the first receiving fibre such that light transmitted from the first transmitting fibre may be received by the first receiving fibre.

15. The combined sensor of claim 14, wherein the distal termini of the first transmitting fibre and the first receiving fibre are separated by an air gap.

16. The combined sensor of claim 14 wherein the distal termini of the first transmitting fibre and the first receiving fibre are separated by a region of optical fibre in which the external cladding has been removed.

17. A combined sensor, suitable for use in direct contact with, or in the vicinity of, a skin surface of a human or animal subject, the combined sensor comprising:
(i) a textile sensor,
the textile sensor comprising a knitted sensor, wherein the knitted sensor is comprised of an electrically conductive yarn that is knitted so as to form a textile that comprises a plurality of stitches that define a stitch pattern, which stitch pattern comprises a measurable electrical contact resistance, wherein the measurable electrical contact resistance varies when external pressure is applied to the textile sensor; and
(ii) an optical sensor,
the optical sensor comprising a fibre-optic reflectance mode photoplethysmography (PPG) sensor.

18. The combined sensor of claim 17, wherein,
the PPG sensor comprises at least a first transmitting fibre having a distal and proximal terminus, wherein the first transmitting fibre is connected to a first light source at its proximal terminus and transmits light from its distal terminus, and a first receiving fibre having a distal and proximal terminus, wherein the first receiving fibre is connected to a first photodetector at its proximal terminus and receives light at its distal terminus;
wherein the distal terminus first transmitting fibre is sufficiently aligned axially or coaxially with the distal terminus of the first receiving fibre such that light transmitted from the first transmitting fibre may be received by the first receiving fibre.

19. The combined sensor of claim 18, wherein the distal termini of the first transmitting fibre and the first receiving fibre are separated by an air gap.

20. The combined sensor of claim 18, wherein the distal termini of the first transmitting fibre and the first receiving fibre are separated by a region of optical fibre in which the external cladding has been removed.

21. The combined sensor of claim 1 for use in a method of monitoring sporting or task orientated performance in a human or animal subject.

22. The combined sensor of claim 1 for use in a method of monitoring clinical signs and/or symptoms in a human or animal patient.

23. The combined sensor of claim 22 wherein the human or animal patient is suffering from a clinical condition or disease selected from the group consisting of: type I or type II diabetes; peripheral vascular disease; cardiovascular disease; kidney disease; hypertension; limb ulcer; and cardiac arrhythmia.

24. A garment comprising the combined sensor combined sensor of claim 1.

25. A wound dressing comprising the combined sensor of claim 1.

26. A method for removing motion artefacts from measurements obtained from a skin surface mounted optical sensor, comprising continually recording applied compression at the site of the skin surface mounted optical sensor and applying a correction to the measurements so as to normalise the measurements and eliminate motion artefacts.

27. The method of claim 26, wherein continual recording of applied compression at the site of the skin surface mounted optical sensor is achieved by combining the optical sensor with a sensor that measures applied compression.
